SEQUENCE LISTING (6 sequences)

SEQ ID NO: 1 - DNA, 24 bp, Artificial Sequence
  Description of Artificial Sequence Synthetic primer
cctcagcgacacaagatgcgatcg

SEQ ID NO: 2 - DNA, 24 bp, Artificial Sequence
  Description of Artificial Sequence Synthetic primer
  modified_base (7): Inosine
  modified_base (9): Inosine
ggttgcntntgaacgttgtgaacc

SEQ ID NO: 3 - DNA, 21 bp, Artificial Sequence
  Description of Artificial Sequence Synthetic primer
gcacgagactagtgagacgtg

SEQ ID NO: 4 - DNA, 20 bp, Artificial Sequence
  Description of Artificial Sequence Synthetic primer
gttttgaacataggtctgcc

SEQ ID NO: 5 - DNA, 21 bp, Artificial Sequence
  Description of Artificial Sequence Synthetic primer
caccgggctcagcgccttcat

SEQ ID NO: 6 - DNA, 20 bp, Artificial Sequence
  Description of Artificial Sequence Synthetic primer
tcgcagcgcatcgccttcta

(19) United States
(12) Patent Application Publication
(10) Pub. No.: US 2007/0061907 A1
(43) Pub. Date: Mar. 15, 2007

(54) Inhibitor of differentiation knockout mammals and methods of use thereof

(76) Inventor: Robert Benezra, Hampton Bays, NY (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 11/300,904

(22) Filed: Dec. 15, 2005

Related U.S. Application Data
(63) Continuation of application No. 10/220,911, filed on Sep. 6, 2002, now abandoned.
(60) Provisional application No. 60/187,893, filed on Mar. 8, 2000.

(51) Int. Cl. A01K 67/027

(52) U.S. Cl. 800/18

(57) ABSTRACT

The invention relates to Id knockout mammals having a disruption in at least one and at most three alleles of inhibitor of differentiation genes, Id1 and Id3. This results in reduction or prevention of a cell proliferative disorder in the mammal as compared to a wild-type mammal. In particular, tumor growth and/or metastasis is shown to be inhibited. Further, tumor growth is shown to have poor vascularization and extensive necrosis in Id knockout mammals lacking 3 out of 4 of the Id1, Id3 alleles (Id1−/−Id3±). Drug screening methods to select agents useful to affect activity or expression of Id1 or Id3 gene products are disclosed. Therapeutic methods employing selected agents in subjects in need of treatment and diagnostic methods and test kits to identify subjects having, or at risk of having, a neurological or cell proliferative disorder are also described.

### CROSS-REFERENCE TO RELATED APPLICATIONS

This application is a continuation of Ser. No. 10/220,911, filed Sep. 6, 2002 which is a National Stage Application of PCT/US01/07378 which claims priority from U.S. Provisional Application Ser. No. 60/187,893, filed Mar. 8, 2000.

### FIELD OF THE INVENTION

The present invention relates to knockout mammals having a disruption in one or more inhibitor of differentiation genes, Id1 and Id3, resulting in reduction or prevention of a cell proliferative disorder in the mammal. In particular, this invention relates to methods of preventing, ameliorating, or treating diseases related to neurogenesis and cell proliferative disorders by agents that affect the activity and/or expression of one or more Id gene products. Drug screening methods to select these agents, and diagnostic methods, and test kits to identify whether a subject has, or is at risk of developing, a neurological or cell proliferative disorder also are described.

### BACKGROUND OF THE INVENTION

Inhibitor of differentiation (Id) genes encode members of the helix loop helix (HLH) family of transcription factors that inhibit transcription by forming inactive heterodimers with basic HLH (bHLH) proteins. There are four members of the Id gene family recognized in mammals, and the proteins they encode share homology primarily in their HLH domain. Typically, bHLH proteins form heterodimers with other bHLH proteins, and their basic domain binds to a DNA sequence element, the E box, activating transcription. Products of Id genes lack the basic DNA binding domain of the bHLH transcription factors, and when they heterodimerize with bHLH proteins, the resultant complexes are inactive. Id proteins play a major role in cell growth and differentiation. Id proteins function at a general level as positive regulators of cell growth and as negative regulators of cell differentiation. The importance of Id proteins as regulatory intermediates for coordinating differentiation linked gene expression has been documented. For a review see, for example, Norton et al., Trends Cell Biol. 8(2):5830 65(1998). Overexpression of Id blocks bBLH-mediated transcription in a wide variety of cell types. Generally, high levels of Id mRNA are detected in proliferative undifferentiated, embryonic cells, and lower levels are detected in well differentiated, mature and adult tissues. In vitro, these genes are generally expressed at lower levels in cells after the induction of differentiation. Thus, it is recognized that Id proteins inhibit differentiation and enhance cell proliferation.

Recently, high levels of expression of Id genes have been identified in cell lines derived from a wide variety of different tumors. Because bHLH complexes regulated by 5 Id may have varied effects on the differentiated state of a cell, overexpression or upregulation of Id has been associated with a block to differentiation (Deed et al., J. Biol. Chem. 27:8278 8286 (1998); Ogata et al., Proc. Nat. Acad. Sci. USA go: 9219 9222 (1993); Neuman et al., Dev. Biol. 160:186 195 (1993)) or an alteration in cell fate (Heemskerk, et al., J. Exp. Med 186:1597 1602 (1997); Martinsen et al., Science 281:988 10 991(1998); Katagiri et al., J. Cell. Biol. 127:1755 1766 (1994)) in different cell contexts.

Id gene expression has been found to be a key mediator of tumor cell biology. See, for example, Israel et al., Cancer Res. 1(59):1726 1730 (1999); and Maruyama et al., Ani. J. Pathol. 155(3):815 22(1999). High incidence of T cell tumors in E2A-null mice and E2A/Id1 double knockout mice is shown to be associated with enhanced proliferative potential of pancreatic cancer cells and of proliferating or dysplastic ductal cells in chronic pancreatitis. Thus, not only the inappropriate proliferation of tumors but also the anaplastic characteristics that contribute to their malignant behavior may be regulated by Id gene expression.

Additionally, it has been shown that Id1 and Id3 are co expressed temporally and 20 spatially during murine neurogenesis and angiogenesis (Duncan et al., Dev. Biol. 154:1-10 (1992); Ellmeier et al., Dev Dvn. 203:163 173 (1995); Jen et al., Dev. Dyn. 208:92 106 (1997)). In general, Id1, Id2 and Id3 are expressed in dividing neuroblasts of the central nervous system (CNS) up to about embryonic day (E) 12.5, after which Id2 expression persists in neurons that are undergoing maturation in both the future cerebellum and 25 cerebral cortex. Id1 and Id3, but not Id2, are expressed in endothelial cells in the brain, whereas Id1, Id2 and Id3 are expressed in endothelial cells throughout the rest of the embryo during development (Jen et al., Dev. Dyn. 208:92 106 (1997), and Lyden et al., Nature 401:670 677 (1999)). In adult animals, Id1 and Id3 are no longer expressed in the brain, but Id2 expression remains in the Purkinje cells of the cerebellum (Neuman et al., Dev. Biol. 16o:186 195 (1993); and Lyden et al., Nature 401:6670 677 (1999)).

It also is known that angiogenesis, the branching and sprouting of capillaries from pre existing blood vessels, occurs in the yolk sac and in the embryo, particularly in the brain (for a review see, for example, Risau et al., Nature 386:671 674 (1997)). Signaling, from both VEGF and Tie 2 receptors has been implicated in this process, as well as in tumor angiogenesis. However, little is known of the involvement of bHLH proteins during, these processes.

In the initial stages of angiogenesis, data suggests that endothelial cells are 5 recruited to tumor sites from neighboring blood vessels (Ogata et al., Proc. Natl. Acad Sci. USA 90: 9219 9222 (1993), Holash, D. & Folkman, J. Cell 86,353 64 (1996), Yankopoulos, G. D. et al. Nature 407 242 8 (2000); Folkman, J. et al. Nature 339,58 61(1989)) and/or from circulating endothelial precursor cells (CEPS). Raffi, S, J. Clin. Invest 10517-19 (2000); Asahara, T. et al, Science, 275, 964 7 (1997); 10 Kalka, C, et al. Oric, Nat'l Acad Sci USA 97 3422 7 (2000); Takahashi, T. et al., Nat Med B5, 434-8 (1999); Peichev, M. et al. Blood 95 952 8 (Feb. 1, 2000)). Several studies have shown that genetically marked bone marrow (BM) cells, when transplanted into the recipient animals can be mobilized to the peripheral circulation, but the phenotype and functional role of these cells in the regulation of angiogenesis has not previously been established.

In view of the devastating effect that can result from neuronal and cell proliferative disorders, there is a need in the art to provide therapeutic methods that can treat, ameliorate, or prevent these disorders through drug design strategies that enable in vitro or in vivo investigation of agents that affect the activity and/or expression of Id gene 20 products in various physiological settings. There is also a need for models which serve to elucidate the functional role of bHLH proteins in cell proliferative disorders. The present invention fulfills these and other needs.

### SUMMARY OF THE INVENTION

The present invention provides novel knockout mammals having a disruption in at least one and at most three alleles of inhibitor of differentiation genes, Id1 and Id3, wherein the disruption results in reduction or prevention of a cell proliferative disorder in the mammal as compared to a wild-type mammal. The knockout mammal can be further genetically transformed with a construct that is capable of producing a cell proliferative 30 disorder spontaneously in the mammal. The construct preferably contains an oncogene or a proto oncogene. Alternatively, a cell proliferative disorder is induced into the mammal via tumor xenografts. According to a preferred embodiment of the invention, the knockout mammal is heterozygous for Id I gene and homozygous for Id3 gene. In one preferred embodiment, the knockout mammal is homozygous for Id1 gene and heterozygous for Id3 gene; most preferably the knockout mammal is Id1−/−, Id3±. Id disruption may affect transcription, 5 translation, and/or post translational modification of a polynucleotide encoding at least one gene product of Id1, Id3, or both.

According to another preferred embodiment of the invention, the cell proliferative disorder comprises cancer induced by tumor xenografts or by genetic transformation in wild type or knockout mammals. Cancer preferably comprises breast cancer, lung cancer, lymphoma, or a combination thereof. Knockout mammals exhibit long term survival rate against these cancers partly because of their inability to vascularize or metastasize tumor cells.

According to another aspect of the invention, there is provided an in vivo or an in vitro system for screening, and evaluating drugs useful in the treatment or prevention of the neuronal and cell proliferative disorders, including tumor vascularization, vasculature mimickery and angiogenesis.

In another aspect, the invention is directed to a method of preventing, ameliorating, or treating a cell proliferative disorder, a neurogenic disorder, or both in a subject in need thereof, comprising administering to the subject a physiologically effective 20 amount of an agent capable of interaction with expression and/or activity of at least one inhibitor of differentiation (Id) gene product in the body of the subject. The subject can be a human subject. Preferably, the agent has an agonistic or an antagonistic affect on expression and/or activity of one or more Id gene products, more preferably the agent antagonizes expression and/or production of one of Id1 or Id3 gene products, most preferably the agent antagonizes activity and/or expression of Id1 gene products.

Additionally, the agent of the invention is administered to an individual suffering from a neurological, cell proliferative disorder, or both through the use of cell or gene therapy techniques. These techniques include, for example, introducing a cell population, preferably the individual's own cells, to the individual, wherein cells have been transformed in vitro with a polynucleotide molecule encoding and expressing in the body of the individual a biologically effective amount of an antagonizer of one or more gene products of Id1, Id3, or both. Preferably, the antagonizer is tetracycline.

According to a preferred embodiment of the invention, Id agonists or antagonists are administered to patients suffering from cancer characterized by inappropriate Id gene products activity and/or expression, along with one or more standard anti cancer drugs, including cytotoxic or chemotherapeutic agents.

Another aspect of the invention features a method to screen agents for use in treating neurological and/or cell proliferative disorders. The screening test is performed in vitro or in vivo. In an in vivo drug screening test, the agent to be tested is administered to a mammal, and the level of expression and or activity of at least one gene product of Id1, Id3, or both is determined in the presence and absence of the test agent. The agent is 10 selected on the basis of its interaction with expression and/or activity of at least one gene product of Id1, 10, or both, as compared to control, for use in treating said neurological and/or cell proliferative disorders. In an in vitro drug screening method, mammalian cells, as opposed to mammals, are employed, and mammalian cells are incubated in the presence and absence of a test agent. The level of expression and/or activity of at least one gene product of Id1, Id3, or both is determined in the presence and absence of the test agent. An agent that interacts with mammalian cell expression and/or activity of at least one gene product of Id1, Id3, or both, is selected for use in treating neurological and/or cell proliferative disorders.

In another aspect, the invention is directed to a diagnostic method of determining whether a subject has, or is at risk for, developing a neurological and/or an angiogenic disorder, comprising the steps of: a) obtaining a sample from the subject; b) determining level of expression and/or activity of at least one gene product of Id1, Id3, or both, in the subject; and b) detecting presence or absence of a genetic mutation in the subject, wherein the genetic mutation results in inappropriate or aberrant one or more Id product activity and/or expression. The genetic mutation identifies a subject that has or is at risk for developing a neurogenic or cell proliferative disorder or disease. Detection of gene mutation is determined using diagnostic test kits which employ probes that specifically hybridize to one or more Id gene or Id gene products. The probes are nucleic acid molecules, or peptides, for example an antibody.

These and other such objects will readily be apparent to one of ordinary skill in the art.

### BRIEF DESCRIPTION OF THE FIGURES

FIG. 1. Tumor growth in wild type and Id knockout mice.

A-C Wild-type (C57BU6/SV129, C57BU6, SV129) and Id knockout mice (Id1±Id3−/−, Id1±Id3+/+ in the mixed genetic background) were injected intradermally with 2×10⁷B6RV2, B-CA breast carcinoma or Lewis Lung carcinoma cell lines as indicated. Tumor surface area was measured every 2 days, and the mean and standard deviations displayed for each group. The number of animals in each group is given in parentheses.

FIG. 2. Vascularization of tumors and metastatic lesions in C57BU6/SV 129 wild-type and Id1±Id3−/− mutant mice.

FIG. 2A (a-d): Macroscopic views of intradermal B6RV2 and LLC tumors. B6RV2 tumors grown in C57BLJ6/SV 129 Id1+/+Id3+/+ (FIG. 2A a) and Id1±Id3−/− (FIG. 2A b) mice are shown in their entirety 6 days post inoculation. Cross sectional view of a LLC tumor recovered from wild type C57BLJ6/SVI29 (Id1+/+Id3+/+) (FIG. 2A c) and mutant (Id1±Id3−/−) (FIG. 2A d) on day 40 post injection demonstrates extensive necrosis and hemorrhage in the mutant.

FIG. 2B (a-d): PECANVCD31 immunostained sections from day 8 control and mutant 20 intradermal B6RV2 tumors reveal numerous blood vessels in the wild type (16±3.2 blood vessels per 200× field) (FIG. 2B a) compared to the mutant (2.7±1.4 blood vessels per 200× frame) (FIG. 2B b). Blood vessels with normal appearing lumens and branches are evident in the control (FIG. 2B c). Stunted vessels with occluded lumens are present in the tumors grown in the Id1±d3−/− mice (FIG. 2B d).

FIG. 2C (a-e): Hematoxylin eosin stained sections are shown for control and wild type intradermal LLC tumor and lung metastasis. On day 20 post injection, viable cells and normal appearing blood vessels are seen for the wild type tumor (FIG. 2C a), in contrast to the appearance of widespread necrosis and hemorrhage in the mutant (FIG. 2C b). Widespread LLC lung metastases are seen in the control by day 20 post injection (FIG. 2C c) with no visible microscopic tumor in all mutant lungs by day 35 (FIG. 2C d, Table 1). An example of one of the few solitary lung nodules found in the Id1 ±Id3+/+ mice is shown in FIG. 2C e, which is relatively small, well-circumscribed and avascular.

FIG. 3. Angiogenesis in wild type and mutant mice following intracomeal injection of 136RV2 lymphoma and LLC cells. Mice of the indicated genotypes were photographed on day post injection with B6RV2 and on day 8 for LLC. Note the 5 extensive blood vessel network formed around the tumors derived from both cell types in the wild type animals (A and C). In contrast, decreased tumor growth (arrows) and a complete absence of a vascular network is noted in the Id1±Id3−/− mutant inoculated with the B6RV2 lymphoma cells (B, arrows). Some growth of the LLC (arrows) appears in the Id mutant with hemorrhages observed (D).

FIG. 4. αv-Integrin and MMP2 staining in vessels of wild type and mutant B6RV2 xenografts.

FIG. 4A (a-f): CD3 1/PECAM, αv-integrin and MMP2 (FIG. 4A-a, c, e,) were visualized by antibody staining to adjacent tumor sections from wild type mice sacrificed on day 8. Stunted blood vessels observed in serially sectioned day 8 tumors from Id1±Id3−/− mice stained positive for CD3 UPECAM but av integrin and MMP2/gelatinase staining are absent (FIG. 4A b, d, f).

FIG. 4B (a-b): Tissues were processed for electron microscopy on day 6 post injection. The width of the extracellular matrix of the blood vessel in the Id mutant animal (FIG. 4B b) is greatly thickened compare to the wild type (FIG. 4B a). Tumor cells are poorly aligned to the extracellular matrix around the blood vessel in the mutant. The blood vessel in the mutant has dense endothelial cell projections into an obstructed lumen. E=endothelial cell T tumor cell; L=lumen of endothelial cell.

FIG. 5. Transplantation of wild type bone marrow rescues tumour growth and vascular channel formation in Id mutant mice.

FIG. 5A: Id mutant or irradiated Id mutant mice reconstituted by Id mutant BM failed to support tumour growth.

FIG. 5B (a-f): Irradiated Id mutant mice rescued with wild type bone marrow (BM) restores tumour growth similar to either wild type or irradiated wild type mice transplanted with the wild type BM. On day 10 after tumor implantation, channel formation is observed in wild type (FIG. 5B a,d), absent in Id mutant (FIG. 5B b,e) and rescued in H&E stained plugs of irradiated Id mutant mice with wild type BM (FIG. 5B c,f).

FIG. 6. BM-derived cells reconstitute the angiogenic defect in Id1±Id3−/− mutant mice.

FIG. 6A(a c). Irradiated Id3(−) mutant mice transplanted with Id3(+) wild type BM were stained for H&E (FIG. 6A b) and PECAM/CD31 (FIG. 6A c) which correspond to Id3 gene expression in darkfield of a blood vessel in a day 14 B6RV2 tumour (FIG. 6A a, arrows).

FIG. 6B (a-f). Transplanted β-gal(−) BM into irradiated wild type hosts failed to stain for LacZ tumours (FIG. 6B a,d) blood vessels, and BM (FIG. 6B d inset). Transplanted β-gal(+) BM stained for LacZ in nearly all vessels in D14 B6RV2 tumours observed in both irradiated Id mutant (FIG. 6B b,e) and wild type (FIG. 6B c,f) recipients, with LacZ+ cells also detected in BM cells (FIG. 6B e,f insets). vWF stains β-gal in blood vessels (FIG. 6B b, inset).

FIG. 7. VEGF induced mobilization is impaired in Id mutant mice. Mobilized peripheral blood mononuclear cells were isolated from AdVEGFI65 treated mice and identified as either VEGFR2+ or CD11b+ cells. The VEGFR2+ CD11b(−) cells were mobilized early on in the wild-type mice, but were nearly undetectable in the Id mutant mice throughout the experimental period.

FIG. 7A. The representative percentages of positive populations in PBMCs are shown.

FIG. 7B. VEGF-mobilized peripheral blood of wild type mice gave rise to late outgrowth endothelial colonies, whereas in Id mutant mice significantly less colonies were detected FIG. 7C (a d). Transplantation of VEGF mobilized PBMC from wild type β-gal+ mice into lethally irradiated Id mutant resulted in the engraftment of the LacZ+ cells and reconstitution of angiogenesis in Id mutant mice. B6RV2 cells implanted for two days onto β-gal+ engrafted BM in Id mutant mice were colonized with BM-derived LacZ+ cells (FIG. 7C a,b). Immunohistochemical analysis of day 2 tumors previously stained for β-gal+ expression demonstrated incorporation of vWF+LacZ+ vessels FIG. 7 C c), decorated by VEGFR1+LacZ+ mononuclear cells (FIG. 7C d). Virtually all the LacZ+ vessels also expressed VEGFR1(FIG. 7Cd).

### DETAILED DESCRIPTION OF THE INVENTION

This invention, as described herein, demonstrates for the first time that inhibitors of differentiation (Id) genes are required to maintain the timing of differentiation in mammalian development, and identifies a role for Id proteins in neurogenic and cell proliferative disorders, including cancer vascularization, vasculature mimickery and angiogenesis, which is of clinical importance.

In one embodiment of the invention, as disclosed herein, it has been demonstrated that at least one copy of the Id1 or Id3 gene is required to prevent embryonic lethality associated with premature neuronal differentiation and angiogenic defects in the brain. The premature neuronal differentiation in the Id1 Id3 double knockout mice indicates that Id1 or Id3 is required to block the precisely timed expression and activation of positively acting bHLH proteins during mammalian development.

Without being limited to any specific mechanism underlying the invention described herein, one possible mechanism is that the premature neuronal differentiation in the Id double knockout mice is due to the inhibition of both the expression and the activity of tissue restricted bHLH proteins by sequestration of E protein heterodimerizing partners. Angiogenesis associated with tumor growth and metastasis in adult animals is shown to be highly sensitive to Id dosage, as even partial loss of Id function results in profound defects in the neovascularization of tumors.

In a general embodiment of the present invention, knockout mammals are generated that are unable to support the growth and metastasis of various types of tumors. In a more preferred embodiment of the invention knockout mammals have a disruption in at least one and at most three alleles of inhibitor of differentiation genes, Id1 and Id3. Most preferably, knockout mammals of the invention are Id1± and Id3−/−. Disruption of Id genes affects, for example, transcription, translation, and/or post translational modification of Id genes.

According to a preferred embodiment of the invention, Id knockout mammals having the genotype of Id1±Id3−/− or Id1±Id3+/+ are generated and shown to fall to support the growth and metastasis of several tumor xenografts. For example, three different tumors fail to grow and/or metastasize in mice lacking 3 out of 4 of the Id1,3 alleles (Id1−/−, Id3±) and any of the tumor growth present show poor vascularization and extensive necrosis.

It is shown that the residual vasculature in the tumors grown in the Id knockout mammals no longer has on its surface αvβ3-integrin or the associated MMP2 metalloproteinase, which normally causes degradation of components of the extracellular matrix. As antagonists of DNA binding factors, Id proteins are required for the expression of genes like αvβ3-integrin or MMP2 by virtue of its ability to sequester transcriptional repressors. Alternatively, the effect of Id on expression of these genes is indirect.

Without being limited to any specific mechanism underlying the invention disclosed herein, one possible mechanism of action for the lack of tumor vascularization in Id knockout mammals of the invention is due to the proteolysis and remodeling of the extracellular matrix, and indeed, a pronounced thickening of the extracellular matrix surrounding endothelial cells in Id knockout mammals occurs.

In one embodiment of the invention, as disclosed herein, it has been demonstrated that either wild type bone marrow (BM) or vascular endothelial growth factor (VEGF) mobilized circulating endothelial precursor (CEP) cells can restore neoangiogenesis in Id1±Id3−/− mice. Transplanted wild-type BM- or VEGF mobilized derived cells were detected throughout the vasculature of inoculated tumors and in vascular channels of VEGF loaded Matrigel plugs. The VEGF driven mobilization of CEPS was completely abolished in Id1±Id3−/− mice. In early phases of revascularization, the tumor vessels were decorated with BM derived mononuclear cells expressing VEGFR1 suggesting that these cells may participate early in post natal angiogenesis. These studies demonstrate that mobilization of VEGF responsive BM derived 30 precursor cells is sufficient for post natal angiogenesis. These mobilized cells by themselves can functionally rescue tumor vasculature in the Id knockout mammals indicating that mature endothelial cells are probably not required for this process.

Furthermore, it was demonstrated that in the initial phases of neoangiogenesis there is infiltration of LacZ+VEGFR1+ BM derived mononuclear cells around developing vessels. Since CEPs and hematopoietic precursor cells, such as myelomonocytic cells, express VEGFRI (Sawano, A., et al. Blood 97, 785 791. (2001), it is possible that both of these cell types are mobilized to the neo angiogenic vascular bed. These studies indicate that BM derived hematopoietic precursor cells as well as CEPS may be required for early neo angiognesis. Some of the VEGFR1+ cells may incorporate into the vessel wall, undergo apoptosis, or recirculate to other neoangiogenic processes such as sites of metastasis.

These studies, utilizing the knockout mammal of the present invention, elucidate the role of BM derived VEGF responsive CEPs in the regulation of post natal angiogenesis and lay the foundation for modulating Id1+/+Id3+/+CEPs to inhibit tumor angiogenesis or to accelerate wound healing. The model would also be useful in further studies aimed at determining whether simultaneous mobilization of hematopoietic precursor cells are essential for incorporation of CEPs.

Without being limited to any specific mechanism underlying the invention disclosed herein, one possible mechanism of action for the lack of tumor vascularization in Id knockout mammals of the invention is that disruption of Id1 and/or Id3 may result in the interference of VEGF receptor signalling, thereby resulting in the failure of CEPs to mobilize to the peripheral circulation.

In accordance with another preferred embodiment of the invention, wild type or knockout mammals are made to spontaneously become oncogenic or cancer prone through genetic transformation by an oncogene or a proto oncogene. Oncogenic mammals of this invention are used, for example, to test physiological interaction between oncogenicity, and inhibitor of differentiation gene products in vivo. For example, an Id knockout and oncogenic mammal is tested for its reduced incidence of neoplasm development, compared to an oncogenic mammal, not having a mutation or disruption of the Id gene.

The knockout mammal of this invention has a variety of uses depending on the Id gene or genes that have been suppressed. Where the Id gene or genes suppressed encode proteins believed to be involved in neurogenesis, the mammal is used to screen for agents useful for neuroaegenerative diseases, for example, agents that either enhance or inhibit the activity and/or expression of one or more Id gene product. Where the Id gene or genes suppressed encode proteins believed to be involved in a cell proliferative disorder, such as cancer or angiogenesis, the mammal is used to screen for agents useful for treating or preventing these disorders.

Knockout oncogenic, or wild-type oncogenic mammals, according to the invention described herein, are used, for example, to test material suspected of being a carcinogen. Such tests are performed by exposing the animal to the material and determining neoplastic growth as an indicator of carcinogenicity. This test can be extremely sensitive because of the propensity of the knockout animals to develop tumors.

The mammals, or cell line derived therefrom, are also used to control the regulation of the bHLH transcription cascade in mammals. The control is achieved, for example, by identifying downstream or upstream-acting chemical regulators of the transcription cascade on the expression of Id gene or loss or gain of expression of other genes. Such studies are achieved by using, for example, chip array analysis of RNA of the Id knockout mammals and comparing the pattern of a particular RNA species to the pattern found in a normal mammal.

This method is especially useful when constitutive expression of bBLH transcription genes that are involved in manifestation of an identifiable phenotype or genotype are deleterious to the growth or health of the mammal. This method is also useful to define distinct states of growth arrest or differentiation, and thereby providing a molecular mechanism coupling growth arrest and differentiation. For example, exit from the cell cycle into a pre-differentiation state of post-mitotic growth arrest can be characterized by changes in the level of the activity or expression of Id genes. Other uses are readily apparent to one of skill in the art.

In another embodiment of the invention, methods of screening agents useful in treating neurological and/or cell proliferative disorders, are disclosed. The screening methods for suitable agents are performed in both an in vitro and in vivo settings. In a preferred embodiment of the invention, agent screening is performed in vitro using mammalian cell culture. In this case, mammalian cells are incubated in the presence and absence of a test agent, and the level of expression and or activity of at least one gene product of an Id gene, for example, Id1, Id3, or both are determined in these cells.

Mammalian cells include, for example, normal mammalian cells, transformed mammalian cells, including those that are made to have aberrant or mutated Id, tumor cells, transformed oncogenic and Id knockout cells, or a combination thereof. An agent that demonstrates interaction with expression and/or activity of one or more Id gene product is selected as a potential drug for treating or preventing a cell proliferative disorder, such as, for example, tumor vascularization or angiogenesis. The same strategy is applied to find compounds that would be useful in suppressing, a neurogenic disorder observed in many patients, if such disorders are accompanied by an aberrant or abnormal production of an Id gene product.

According to a more preferred embodiment of the invention, agent screening is preformed in vivo using test and control mammals. Depending on the type of agents to be screened and the desired affect, various mammals are used as test or control. For example, wild-type mammals, knockout mammals, knockout and oncogenic mammals, knockout and tumor xenografts mammals, wild-type and oncogenic mammals, wild-type and tumor xenografts mammals, or a combination thereof are used in an agent screening test.

Included within the scope of this invention are methods for preventing., ameliorating, or treating a cell proliferative disorder, a neurogenic disorder, or both in patients. These lo methods comprise administering to the patients a physiologically-effective amount of an agent capable of interaction with expression and/or activity of at least one inhibitor of differentiation (Id) gene product in the body of the patient.

According to a more preferred embodiment of the invention, interaction between the agent and one or more Id gene products is an antagonistic interaction. For example, patients suffering from cancer may experience an elevated level of an Id protein. It would be desirable to identify therapeutic agents that reduce the level of Id proteins, which in turn reduce or eliminate vascularization, and/or metastasis of tumor, by administering to the patient a therapeutic agent capable of producing such effects.

In general, unwanted cell proliferation results from inappropriate Id protein expression and/or activity. Id proteins are expressed in different types of cells including cancer cells, cells surrounding a cancer cell (stromal cells), endothelial and smooth muscle cells. For example, an increase in Id protein activity of endothelial cells surrounding cancer cells may lead to an increased vascularization of the tumor, thereby facilitating growth of the cancer cells by angiogenesis.

Alternatively, or as well as tumor angiogenesis, an increase in Id activity may generate vasculature channels that facilitate tumor perfusion independent of tumor angiogenesis. This phenomenon, which is referred to as “vasculature mimickery” (see, Maniotis et al., Am. J Pathol.155(3):739-52 (1999) incorporated herein by its entirety) facilitates regeneration of vasculature channels that assist tumor perfusion in highly invasive tumors. It has been found that neither normal melanocytes nor poorly invasive melanoma cells generated these patterned channels in vitro under identical culture conditions, even after the addition of conditioned medium from metastatic pattern forming melanoma cells, soluble growth factors, or regimes of hypoxia.

Thus, inappropriate Id protein activity can contribute to a cell proliferative disorder in different ways such as, for example, through increasing the production of growth factors, causing aberrant growth of a cell, and increasing formation and spreading of blood vessels in solid tumors thereby supporting tumor growth.

Included within the scope of this invention are diagnostic methods for determining whether a subject has, or is at risk of developing, a neurological and/or an angiogenic disorder. The method comprises a) obtaining a sample from a subject b) determining level of expression-and/or activity of at least one gene product of Id1, ID3, or both; in said subject; and b) detecting, presence or absence of a genetic mutation in the subject, wherein the genetic mutation comprises an alteration in the activity and/or expression of at least one gene product of Id1, Id3, or both. The presence of a genetic mutation in one or more of the Id genes or gene products identifies a subject that has, or is at risk for developing, a neurogenic or cell proliferative disorder or disease.

Based on known nucleotide sequences of human Id genes, one of ordinary skill in the art, employing the techniques for genotyping mice according to the invention disclosed herein, can easily design nucleotide primers for human Id genes and use the primers to detect a mutation in one or more Id genes of a human.

Further included within the scope of this invention are methods for reducing or inhibiting tumor vasculature in a subject through cell and/or gene therapy techniques. One such technique requires, for example, introducing a nucleic acid molecule, by a vector, on its own or as integrated in transformed cells, to an individual, wherein the nucleic acid molecule encodes or affects production of gene products that can interact with one or more Id gene products in vivo. Preferably, the antagonizer is tetracycline. According to a more preferred embodiment of the invention, the transformed cells are the individual's own cells, and upon administering to the individual, stably express therapeutic Id gene products within the individual's body.

As used herein “Cell proliferative disorders” refer to disorders wherein unwanted cell proliferation of one or more subset of cells in a multicellular organism occurs resulting in harm (e.g., discomfort or decreased life expectancy) to the multicellular organism Cell proliferative disorders occur in different types of animals and in humans, and include cancers, blood vessel proliferative disorders, and fibrotic disorders.

As used herein “Inappropriate or aberrant Id product activity and/or expression includes, for example, any change in the activity and/or expression of an Id product, as compared to the normal activity and/or expression of the Id product, including, for example, Id protein expression in cells which normally do not express Id protein; lack or reduction of Id protein expression in cells which normally do express Id protein; increased Id protein expression resulting, in unwanted cell proliferation or mutations leading to constitutive activation of Id protein; a change in the molecular structure of one or more Id genes, or gene products; reduction of Id protein activity and/or expression leading to excessive cell differentiation. The existence of inappropriate or aberrant Id product levels or activities is determined by procedures well known in the art.

“Id product”, Id gene product”, or “Id protein” is used interchangeably herein and includes any protein, peptide, polypeptide, polynucleotide, in sense or antisense orientation.

As used herein, “physiologically effective amount” refers to an amount capable of producing an “affect” on the production and/or activity of at least one Id gene product.

The term “affect” is defined broadly herein and encompasses any type of interaction, including, but not limited to antagonistic or agonistic interactions. Furthermore, agents having both antagonistic and agonistic affect on one or more Id gene products are also included within the scope of the invention.

The “agent” of this invention includes any compound, composition or small molecule that interacts with activity and/or expression of one or more inhibitor of differentiation gene product(s) in vitro, ex vivo, or in vivo. The agents can be, for example, any protein, peptide, polypeptide, nucleic acid molecule, including DNA, RNA, DNA/RNA hybrids or an antisense molecule, small molecules, antibiotics, and the like.

The agent, according to the invention, is used to treat a cell proliferative or a neurogenic disorder by administering a therapeutically effective amount of the agent to a patient in need thereof.

The agent also is used in vitro studies to investigate the mechanism of action of the Id proteins, and interaction between Id and other genes and gene products in the angiogenic cascade in various clinical setting.

As used herein, the term “knockout” includes, for example, a partial or complete suppression of the expression of at least a portion of a product encoded by an endogenous DNA sequence in a cell. Preparation of a knockout mammal can be achieved by methods known in the art. For a review see, for example, Pfeffer et al., Cell 73:457-467 (1993)) which describes mice in which the gene encoding the tumor necrosis factor receptor p55 has been suppressed. These mice showed a decreased response to tumor necrosis factor signaling. Fung-Leung et al, Cell 65:443-449 (1991); J. Exp Med, 174:1425-1429 (1991)) describe knockout mice lacking expression of the gene encoding CD8. These mice were found t o have a decreased level of cytotoxic T cell response to various antigens and to certain viral pathogens such as lymphocytic choriomeningitis virus.

The knockout mammal of this invention is made, for example, by introducing a nucleic acid construct that suppresses expression of an Id1 or Id3 gene into an undifferentiated cell type, such as embryonic stem cell. This cell is then injected into a mammalian embryo, where it then is integrated into the developing embryo. The embryo is then implanted into a foster mother for the duration of gestation.

Knockout mammals are typically produced by introduction of a knockout construct into the genome of the mammal. “Knockout constructs” encompass nucleic acid sequences that are designed to decrease or suppress expression of a protein encoded by endogenous DNA sequences in a cell. The nucleic acid sequence used as the knockout construct is typically comprised of (1) DNA from some portion of the Id gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (Le., regions of the knockout construct that are homologous to endogenous )DNA sequences hybridize to each other when the knockout construct is inserted into the cell and recombine so that the knockout construct is incorporated into the corresponding position of the endogenous DNA). The knockout construct nucleic acid sequence may comprise 1) a full or partial sequence of one or more exons and/or introns of the Id gene to be suppressed, 2) a full or partial promoter sequence of the Id gene to be suppressed, or 3) combinations thereof.

As used herein “disruption of the gene” and “gene disruption” include, for example, insertion of an exogenous nucleic acid sequence into one region of the native DNA sequence (usually one or more exons) and/or the promoter region of a gene so as to decrease or prevent expression of that gene in the cell as compared to the wild-type or naturally occurring sequence of the gene. When a construct containing this exogenous nucleic acid sequence is transfected into a cell, the construct integrates into the genomic DNA.

The term “progeny” refers to any and all future generations derived and descending from a particular mammal, i.e., a mammal containing a knockout construct inserted into its genomic DNA. Thus, progeny of any successive generation are included herein such that the progeny, the F1, F2, F3, generations and so on (indefinitely) are included in this definition.

Included within the scope of this invention is a mammal in which two or more genes have been knocked out. Such mammals can be generated by repeating the procedures set forth herein for generating each knockout construct, or by breeding mammals, each with a single gene knocked out, and screening for those with the double or single knockout genotypes. This procedure is defined as “intercrossing”, herein.

The DNA sequences to be used to knock out a selected gene are obtained using methods well known in the art such as those described by Sambrook et al., Molecular Cloning: A Laboratory Manual, Cold Spring Harbor Laboratory Press, ColdSpring Harbor, N.Y. (1989)). Such methods include, for example, screening a genomic library with a cDNA probe encoding at least a portion of the same gene in order to obtain at least a portion of the genomic sequence. Alternatively, if a cDNA sequence is to be used in a knockout construct, the cDNA is obtained by screening, a cDNA library with oligonucleotide probes or antibodies (where the library is cloned into an expression vector). If a promoter sequence is to be used in the knockout construct, synthetic DNA probes are designed for screening a genomic library containing the promoter sequence.

Another method for obtaining the DNA to be used in the knockout construct is to manufacture the DNA sequence synthetically, using a DNA synthesizer.

The DNA sequence encoding the knockout construct must be generated in sufficient quantity for genetic manipulation and insertion into embryonic stem (ES) cells.

Amplification is conducted by 1) placing the sequence into a suitable vector and transforming bacterial or other cells that can rapidly amplify the vector, 2) by PCR amplification, or 3) by synthesis with a DNA synthesizer.

This invention farther contemplates production of knockout mammals from any species of rodent, including without limitation, rabbits, rats, hamsters, and mice. Preferred rodents include members of the Muridae family, including rats and mice. Generally, the embryonic stem cells (ES cells) used to produce the knockout mammal will be of the same species as the knockout mammal to be Generated. Thus, for example, mouse embryonic stem cells will usually be used for Generation of knockout mice.

Embryonic stem cells are typically selected for their ability to integrate into and become part of the germ line of a developing embryo so as to create germ line transmission of the knockout construct. Thus, any ES cell line that is believed to have this capability is suitable for use herein. One mouse strain that is typically used for production of ES cells, is the 12₉J strain. The cells are cultured and prepared for DNA insertion using methods well known to the skilled artisan such as those set forth by Robertson in: Teratocarcinonuzs and Embryonic Stem Cells: A Practical Approach, E. J. Robertson, ed. IRL Press, Washington, D.C. (1987); Bradley et al., Current Topics in Devel. Biol, 20:357-371(1986); and Hogan et al., Manipulating the Mouse Embryo: A Laboratory Manual, Cold Spring Harbor Laboratory Press, Cold Spring Harbor, N.Y. (1986).

Insertion of the knockout construct into the ES cells is accomplished using a variety of methods well known in the art, including for example, electroporation, microinjection, and calcium phosphate transformation.

For insertion of the DNA sequence, the knockout construct DNA is added to the ES cells under appropriate conditions for the insertion method chosen. Where more than one construct is to be introduced into the ES cell, DNA encoding each construct can be introduced simultaneously or one at a time.

Screening is done using a variety of methods. When the marker gene is an antibiotic resistance gene, the cells are cultured in the presence of an otherwise lethal concentration of antibiotic. Those cells that survive have presumably integrated the knockout construct. If the marker gene is other than an antibiotic resistance gene, a Southern blot of the ES cell genomic DNA is probed with a sequence of DNA designed to hybridize only to the marker sequence. Finally, if the marker gene is a gene that encodes an enzyme whose activity can be detected (e.g., beta-galactosidase), the enzyme substrate is added to the cells under suitable conditions, and the enzymatic activity is analyzed.

The knockout construct is integrated into several locations in the ES cell genome, and integrates into a different location in each cell's genome, due to the occurrence of random insertion events. The desired location of the insertion is in a complementary position to the DNA sequence of Id1 or Id3 genes. Typically, less than about 1-5 percent of the BS cells that take up the knockout construct actually integrate the knockout construct in the desired location. To identify those cells with proper integration of the knockout construct, the DNA is extracted from the cells using standard methods such as those described by Sambrook et al., supra. The DNA is then probed on a Southern blot with a probe or probes designed to hybridize in a specific pattern to genomic DNA digested with (a) particular restriction enzyme(s). Alternatively, or additionally, the genomic DNA is amplified by PCR with probes specifically designed to amplify DNA fragments of a particular size and sequence (i.e., only those cells containing the knockout construct in the proper position generate DNA fragments of the proper size).

After suitable ES cells containing, the knockout construct in the proper location have been identified, the cells are inserted into an embryo. The suitable stage of development for the embryo is very species dependent. However, for mice it is about 3.5 days. The embryos are obtained, for example, by perfusing the uterus of pregnant females. Suitable methods for accomplishing this are known to the skilled artisan, and are set forth by Bradley et al., supra.

After ES cells have been introduced into the embryo, the embryo is implanted into the uterus of a pseudopregnant foster mother. While any foster mother maybe used, they are typically selected for their ability to breed and reproduce well, and for their ability to care for their young. Such foster mothers are typically prepared by mating with vasectomized malts of the same species. The stage of the pseudopregnant foster mother is important for successful implantation, and it is species dependent. For mice, this stage is about 2-3 days pseudopregnant.

Offspring that are born to the foster mother are screened for the presence of the knockout construct using Southern blots and/or PCR as previously described. Intercrossing is achieved, for example, by crossing offspring that possess the construct to each other, if they are believed to carry the knockout construct in their germ line, to generate homozygous knockout animals. If it is unclear whether the offspring will have germ line transmission they are crossed with a parental or other strain and the offspring screened for heterozygosity. The heterozygotes are identified by Southern blots and/or PCR amplification of the DNA, as set forth above.

The heterozygotes then are crossed with each other to generate homozygous knockout offspring. Homozygotes are identified by Southern blotting of equivalent amounts of genomic DNA from mice that are the product of this cross, as well as mice that are known heterozygotes and wild type mice, Probes to screen the Southern blots are designed as set forth hereinabove.

Other means of identifying and characterizing the knockout offspring are available. For example, Northern blots are used to probe the mRNA for the presence or absence of transcripts encoding either the Id1 or Id3 genes, the marker gene, or both. In addition, Western blots are used to assess the level of expression of the Id1 or Id3 genes in various tissues of these offspring by probing the Western blot with an antibody against Id1 or Id3 protein encoded by the gene knocked out, or an antibody against the marker gene product, where this gene is expressed. Finally, in situ analysis (such as fixing the cells and labeling with antibody) and/or FAGS (fluorescence activated cell sorting) analysis of various cells from the offspring are conducted using suitable antibodies to look for the presence or absence of the knockout construct Id gene product.

As used herein, an “oncogene” or “proto-oncogene”, includes, for example, a polynucleotide which, when incorporated into the genome of the animal, increases the probability of the development of neoplasm (particularly malignant tumors) in the animal. There are several means by which an oncogene is introduced into an animal embryo (See, for example, U.S. Pat. No. 4,736,866, incorporated herein by its entirety). One such method is to transfect the embryo using a vector containing an already translocated oncogene. Other methods involve modifying the oncogene or its control sequences prior to introduction into the embryo. Other methods use an oncogene whose transcription is under the control of a synthetic or viral activating promoter, or to use an oncogene activated by one or more base pair substitutions, deletions or additions.

According to a preferred embodiment of the invention, oncogene sequences are introduced at the fertilized oocyte state to ensure that the oncogene sequence will be present in all of the germ cells and somatic cells of the knockout animal. Introduction of the oncogene sequence at a later embryonic stage might result in the oncogene's absence from some somatic cells of the founder animal, but the descendants of such an animal that inherit the gene will carry the activated oncogene in all of their germ cells and somatic cells.

An oncogene, or proto-oncogene encompasses any foreign sequences or any homologous endogenous sequences, and includes for example, K-ras, Ha-ras, and c-myc. Qncogenes can be placed under the regulatory control of, for example, Mouse Mammary Tumor Virus (MMTV) or Rous Sarcoma Virus (RSV) viral promoter sequences, or the like.

The invention will be more fully understood by reference to the following examples. These examples are not to be construed in any way as limiting the scope of this invention. All literature cited herein is specifically incorporated by reference.

### EXAMPLES

### Example 1

### Id1-l-16-l-Mice

Mice lacking one to four alleles of Id1 and Id3 are generated by intercrossing Id1+/Id3± mice. Offspring lacking one to three alleles in any combination are indistinguishable from the wild type, but no animals lacking all four Id alleles are born. To determine when Id1−/−Id3−/− mice die, Id1−/Id3± mice are intercrossed and the embryos examined from E8.5 to birth. Between E8.5 and E 10.5, Id1−/−Id3+/+, Id1a-Id3± and Id14-Id3-I-embryos are represented in a 1:2:1 mendelian ratio. Id1−/Id3−/− embryos are grossly normal up to E10.5 but reduced in size by 30% at E11.5 and E12.5. By E12.5, the mutants exhibit cranial hemorrhage and no embryos survive beyond E13.5, indicating that expression of either Id1 or Id3 is essential for viability.

The ganglionic eminences of Id1−/−Id3−/− embryos develop cavitational lesions. Areas of hypocellularity form at E11.5, and by E12.5 coalesced into a cavity. Aberrant capillaries flank the cavity, which rupture at E13.5, resulting in hemorrhage throughout the ventricular system.

### Example 2

### Id Inhibits Neuronal Differentiation

Mice having Id1−/−Id3−/− mutation are examined to determine the rate of growth and differentiation of their brain cells. It is found that Id1−/−Id3−/− mutant mice have smaller brain size than the wild-type mice. The decrease in the brain size of the mutant mice may be due to a decrease in proliferation of neuroblasts, or an increase in apoptosis.

At E10.5, no significant differences in apoptosis are observed between wild-type and Id1−/−Id3-1-embryos. Similarly, at E10.5, no difference is found when cell proliferation was measured by immunodetection of K-67, a nuclear antigen expressed in all proliferating cells except those in C30 or early G1. In El 1.5 mutants, however, there are fewer proliferating cells in the neuroepithelium of the telencephalon and the rhombencephalon.

The withdrawal of neuroblasts from the cell cycle is accompanied by altered expression of cell cycle regulatory proteins. In Id1-null embryonic fibroblasts, p16, the cycline D/cdk4 inhibitor, is upregulated. Consistent with this observation, p16 and p27 are upregulated in the mutant neopallial cortex at E11.5. The expression patterns of post-mitotic differentiation markers, microtubule-associated protein 2 (MAP2) and unique Btubulin JUJI), are complementary to that of Ki-67.

### Example 3

### Regulation of Neuronal bHLH Expression By Id

To define molecular mechanism of premature neuronal differentiation in the mutants, the expression pattern of neuronal-specific bHLH genes is examined. Like myogenic bHLH proteins, a hierarchy of activities is found in the neuronal bHLH family within the determination genes (MATH 1, MATH3, MASH-1, Ngn1 and Ngn2) including the expression of differentiation effectors (NeuroD1, NeuroD2 and MATH2) (Risau et al., Nature 386: 671-674, (1997)).

Neuro DI expression is similar in wild-type and mutant embryos at E9.5 and E10.5, but is increased in the ganglionic eminences at E11.5. No differences are observed in NeuroD2 and NeuroD3 expression. In E11.5 mutants, MATH1 expression is more extensive in the rhombencephalon. MATH2 is enhanced and more extensive in the ganglionic eminences and dorsal rhombencephaion, and MATH3 is more extensive in the dorsal telencephalon and ganglionic eminences. By E12.5, no differences are detectable for these markers. In the ventral telencephalon, the areas of premature MATH3 expression are more restricted than those of MATH2 and NeuroD1.

No changes in the expression of BF-I (Xuan et al., Neuron 14:1141-1152 (1995), Dix-2 (Price et al., Nature 351:748-751(1991)), Emx-2 (Simeone et al., EMBO J. 12:2735-2747 (1993)), Pax-6 (Walther et al., Development 113:1435-1449 (1991)) and Shh (Shimamura et al., Development 121:3923-3933 (1995)) are observed, indicating that premature lo neurogenesis in the mutants is not the result of alterations in telencephalic patterning. These observations, the overlapping expression of Id1 and Id3 in the neuroepithelium, and the lack of a neuronal phenotype in Id1 or Id3 single knockout mice indicate that either Id1 or Id3 is required for normal neurogenesis.

### Example 4

### Identification of Vascular Defect in the Braun

Aberrant endothelial cells observed in the ganglionic eminences of Id1−/−Id3−/− embryos express CD31 normally. They form enlarged, dilated blood vessels at E11.5 and an anastomotic network at E12.5. Laminin and fibronectin are expressed normally in the basement membrane. By laminin immunostaining, an absence of branching and sprouting of capillaries into the neuroectoderm of the ganglionic eminences is detected, demonstrating abnormal angiogenesis in the mutants. This is consistent with expression of Id1 and IM in normal vasculature in the CNS during development. Although Id1, Id2 and Id3 are expressed in blood vessels outside the CNS, Id2 expression is absent from blood vessels in the CNS.

At E12.5, reduced expression of vascular endothelial growth factor (VEGF), Flk-1 (VEGF receptor 2) and smooth muscle action is first noted within the vascular malformation, but not in blood vessels outside the ganglionic eminences in the mutants. Vasculogenesis is normal in the mutants. The formation of the major vessels, intersomitic vessels, perineuronaI vascular plexus and endocardium is unimpaired, and yolk sac blood islands of E8.5 mutant embryos are normal.

The cavitational lesions in Id1−/−Id3−/− nice resemble the defect in αv-integrin-null mice. However, no differences in the expression of Id1 or Id3 are observed in av integrin null mice. In addition, differences in the phenotypes of these mice are noted. Capillary sprouts are present in the neuroectoderm of αv-integrin null mice unlike Id mutants, indicating that the angiogenic defect in Id1−/−Id3−/− mice may not be an obligate consequence of hypocellularity. Also, αv-integrin null embryos display only dilated, not malformed blood vessels.

### Example 5

### Reduced Id Dosage Inhibits Growth

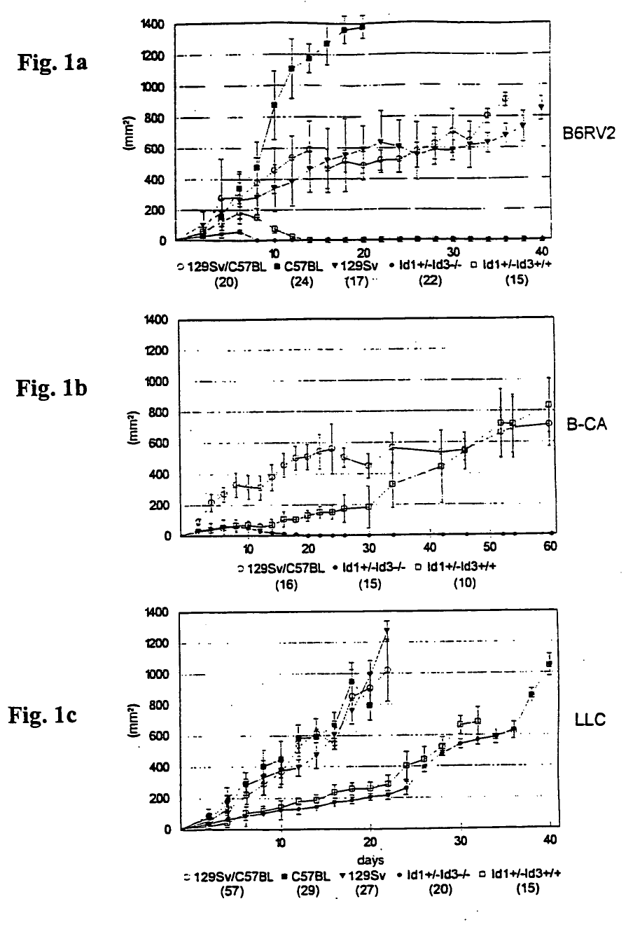

In order to determine if partial loss of Id function alters the vascularization and growth of tumors, Id1±Id3−/− mice and wild-type littermates are challenged with intradermal injections of various tumor cell lines. Wild-type Mice (129Sv/C5TBIJ6) inoculated with the B6RV2 lymphoma cell line show a rapid increase in tumor mass which result in death at 24.1±7.1 days when averaged over 20 animals (FIG. 1A, Table 1). At the tinge of death, all wild-type animals show evidence of Metastasis in the mesenteric lymph nodes by gross inspection (Table 1). These results are independent of the genetic background of the wild-type mice as pure 129Sv or C57BI/6 mice as well as mixed Sv129IG57BL/6 mice all show a similar response to the tumor cell implants (FIG. 1A). A similar result is observed when wild-type mice are inoculated with a murine breast cancer cell line (B-CA), although this line displays a lower metastatic potential (FIG. 1B, Table 1).

In striking contrast, the Id1+I-10-1-mice are completely resistant to the growth and metastasis of the B6RV2 Lymphoma cells and the B-CA breast cancer cells. At 6 and 8 days post inoculation there is a small peak in growth of the B6RV2 and B-CA cells, respectively (FIG. 1A,B). However, by 10 and 20 days post-injection the small masses have completely regressed and no tumor reappears for the duration of the experiment (FIG. 1AB). All 22 mice inoculated with B6RV2 cells remain healthy for 540 days after which time they are sacrificed and examined for evidence of lymph node involvement. None of the animals shows any histological evidence of metastasis in mesenteric lymph nodes (Table 1).

TABLE 1
Outcome of Tumor Cell Implantation Experiments

| | | Overall Survival | Survival Duration | Metastases |
|---|---|---|---|---|
| Tumor Cell Type | Genotype | (total animals) | (days) | (total animals) |
| B6RV2 | Id1 + l − Id3 − l− | (22122) | ⁴540 | (00/22)M |
| | Id1 +/− Id3+/+ | (15115) | ⁴540 | (0115)M |
| | Id1 + I + Id3 + l+ | (0/20) | 24.1 ± 7.1 | (20/20)G |
| | | | | [28.1 ± 8.2] |
| ²B-CA | Id1 + I − Id3−/− | (15115) | ⁴330 | (0/15)M |
| | Id1 +/− Id3+/+ | (OI10) | ⁴60 | (0/10)M |
| | Id1 +/+ Id3+/+ | (0116) | ⁴60 | (2/16)G [l] |
| ³LLC | Id1 +/ Id3 − 1− | (0120) | 4.0.2 ± 2.3 | (0/20)M |
| | Id1 + I − Id3+/+ | (0115) | 34.2 ± 3.7 | (3/15)M |
| | Id1 + I + Id3+/+ | (0157) | 18.93.6 | (57/57)G |
| | | | | [7.9 ± 4.2] |

1 = Mesenteric lymph node metastases
²= Breast Cancer cells
³= Lung metastases
⁴= sacrificed
G = gross and
M = microscopic disease
[ ] = total mets/anim.

All 15 mice, inoculated with the B-CA line also remain healthy for the duration of the experiment (330 days) again with no evidence of metastasis (Table 1)

When the dosage of Id protein is increased in mice, an intermediate phenotype with respect tom tumor resistance is observed. Id1±Id3a+ mice inoculated with lymphoma cells show more robust tumor growth at 6 days post inoculation relative to the Id1±Id3-1− mice and a 4-6 day delay in the time to total regression (FIG. 1A). In addition, unlike the Id1±Id3-1− mice, the Id1±Id3+/+ mice support tie eventual growth of the B-CA cell line although the rate of tumor growth is significantly delayed relative to the wild-type controls (FIG. 1B).

A third tumor cell line, Lewis lung carcinoma (LLC), is used to challenge the Id 20 knockout mice in the xenograft assay. Unlike the B6RV2 or B-CA cells, the LLC cells continue to proliferate in all strains of mice, albeit at a significantly lower rate in the Id1±Id3 I− and Id1±Id3+/+ mice relative to the wild-type littermates (FIG. 1C). As in the case of B6RV2, the growth rate of the LLC in wild type mice is the same in pure 129Sv and C57BL or mixed genetic backgrounds. The survival time of the Id1∓Id3−/mice supplanted with LLC cells is greater than twice that of the wild-type mice (with the Id1±Id3+/+ mice showing an intermediate phenotype (Table 1). This difference in survival time may be accounted for by the absence of metastatic lesions observed in the Id knockout mice relative to the controls (Table 1) and/or differences in the histology of the tumors.

### Example 6

### Angiogenic Defects in Tumors

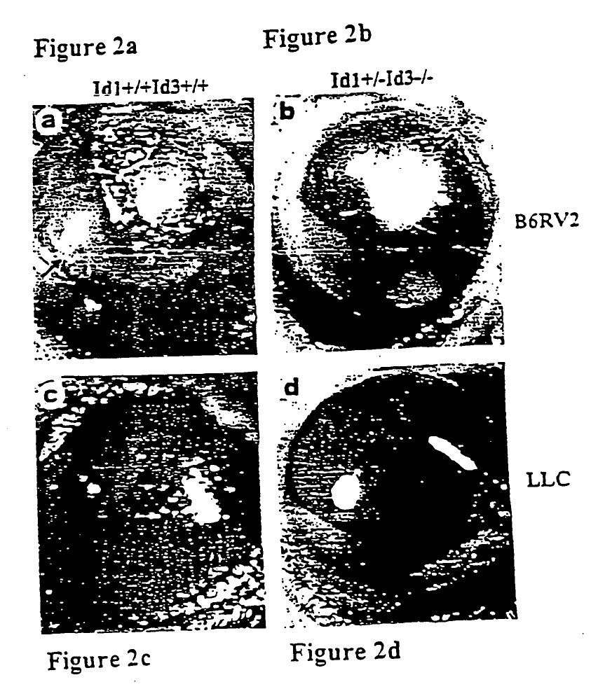

Animals implanted with the B6RV2 lymphoma cells and LLC cells are sacrificed after 6 and 20 days, respectively and the site of injection examined by gross morphology and histology (FIG. 2A-D. Wild-type animals inoculated with B6RV2 show clear evidence of tumor cell growth and blood vessel infiltration after 6 days. In the Id1±Id3−/− animals however, after 6 days while there appears to be some growth of the tumor cells, little if any vascularization of the mass is observed. A cross section of a primary LLC tumor grown in the wild-type animals for 20b days showed a normal appearing cellular mass with atypical tumor vascular bed. LLC cells grown in the Id1±Id3−/− animals, however, consistently produce a primary mass composed primarily of necrotic tissue and hemorrhage.

A typical histological section of an 8 day B6RV2 cell tumor grown in the wild-type background stained with CD3 I/PECAM shows clear evidence of normal appearing capillaries with wide lumens and branching. In the Id1±Id3−/− animals however, after 6 days while there appears to be some growth of the tumor cells, little, if any, vascularization of mass is observed These tumors contain a reduced number of blood cells, and these vessels appear stunted or occluded relative to the controls. Similarly, histological analysis of sections from LLC tumors confirm the appearance of normal blood vessels throughout the tumor grown in wild-type animals. In tumors grown in the Id1+/104− mice however, few normal. blood vessels are observed with evidence of widespread hemorrhage and necrosis. Viable tumor cells are seen only at the peripheral margins.

### Example 7

### Tumor Metastasis

The mutant mice Id1±Id3−/− and wild-type mice are implanted with LLC and examined far the evidence of tumor metastasis. A striking difference is observed between the wild-type and Id mutant mice when metastasis to the lungs is examined. 51!57 of the wild-type animals show evidence of lung metastasis by gross inspection at the time of death (avg. of 7.9+4.2 nodules) whereas 0/20 of the Id1±Id3−/− mice show such lesions (Table 1). In addition, none of the Id1±104 animals shows evidence of Microscopic disease in the lung histologically whereas metastatic lesions with blood vessel infiltration in the wild-type control animals are readily apparent. Once again, the Id1+l−Id3+1+ animals show an intermediate phenotype with 3115 animals possessing microscopic lesions in the lung. Interestingly, these lesions are well encapsulated and show no blood vessel infiltration. Although the Id knockout animals inoculated with 'LLC cells eventually died by day 40 on average (see Table 1), the explanation for this remain unclear. The results suggest that the cause of death is not related to the widespread metastatic lung disease observed in the wild-type mice.

### Example 8

### Mode of Id Action in Tumor Angiogenesis

In order to determine if the failure of the LLC cells to metastasize in the Id knockout animals is solely due to a failure of the cells in the primary lesion to enter the bloodstream (perhaps due to the defect in the vascular bed observed), LLC cells are injected into the tall veins of wild-type and Id1±Id3−/− mice and the appearance of metastases to the lungs quantitated. The wild-type animals display extensive metastatic disease in the lung with blood vessel infiltration after 8 days (6/6 animals tested). In sharp contrast, in the Id1±Id3−/− group, 5/6 animals show no evidence of lung metastasis after 21 days with the remaining animal showing an avascular, well encapsulated tumor nodule.

From these analyses, it is concluded that the failure of MC cells to metastasize to the lungs is not only due to a failure of the cells in the primary tumor to transmigrate through the endothelial cells, but rather the homing and/or establishment of the tumor cells in the lungs is also defective in these animals.

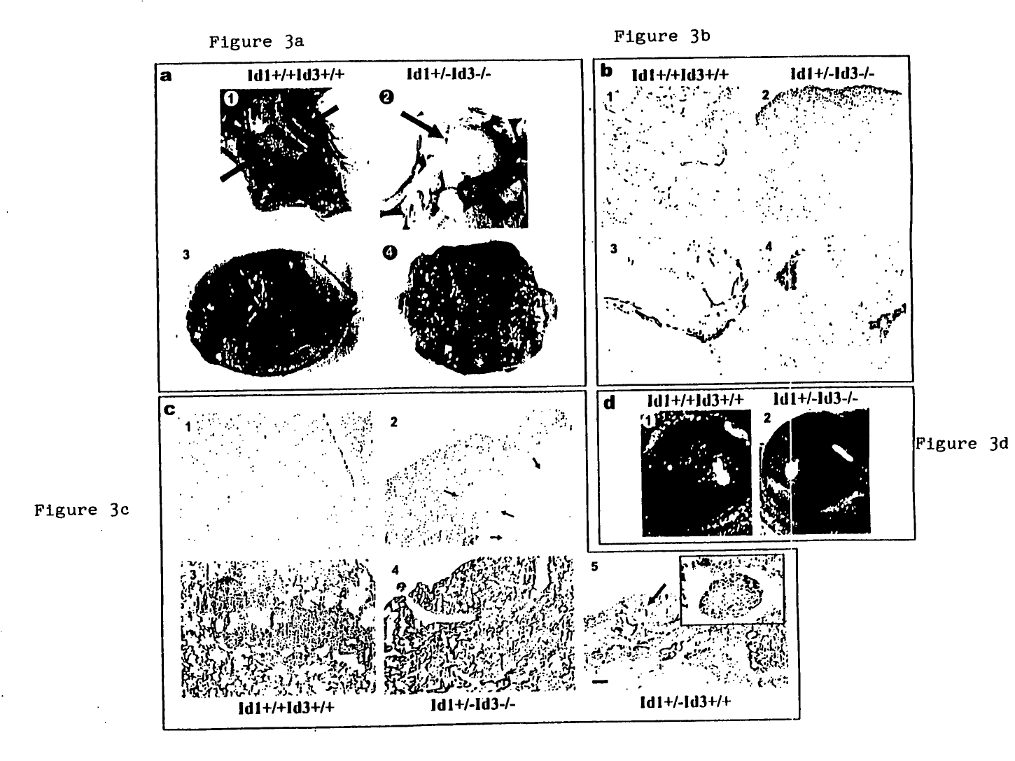

To rule out the possibility that the failure in tumor growth in the id knockout animals is due to an anti-tumor immune response, tumor cells are implanted into the corneal layers of the eye which are avascular and therefore have no inherent cell mediated immunity. Both the B6RV2 line (after 21 days) and the LLC cells (after 8 days) show extensive growth in the wild-type animals (FIG. 3A). In both cases, extensive growth of blood vessels into the tumor mass is observed. In sharp contrast, when this experiment is performed on the Id1±Id3−/− mice, minimal tumor growth of the B6RV2 is observed after 21 days with little or no vascularization of the tumor mass (FIG. 3B; 6 animals tested). Similarly, the ]LLC cells grow to a minimal size in the Id1±Id3−/− mice after 8 days with no clear vascular network development (FIG. 3D; 6 animals tested). Some hemorrhage is observed in the region of the tumor mass in these animals.

The results show that there is no cellular immune infiltration in the tumors or tumor remnants in the Id knockout animals and the growth of the tumors is independent of the genetic background (see FIGS. 1-4). In addition, ablation of NK cells with an anti-asialo antibody in the Id1, 3Ko animals fails to restore the ability of the lymphoma cell line to grow or metastasize.

### Example 9

### Association Between MMP2, αvβ3-Intergin and Angiogenesis

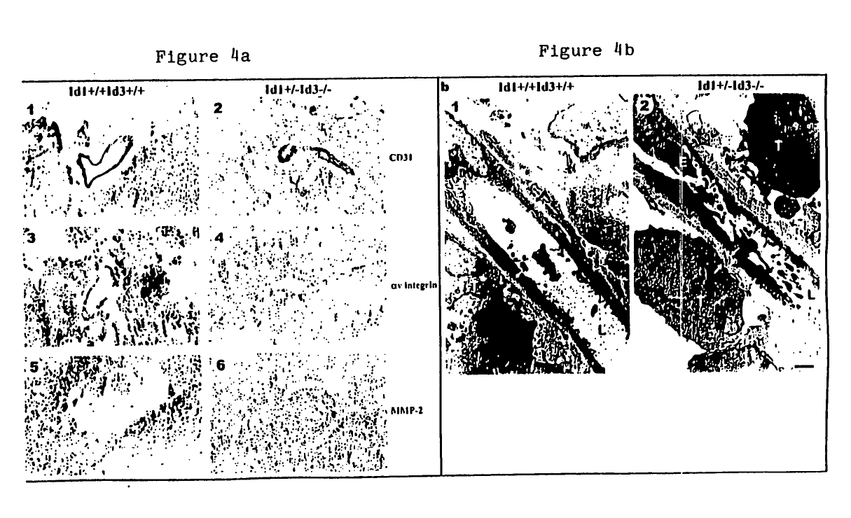

The levels of αv-integrin and MMP2 on the endothelial cells of the tumors grown in the wild-type and Id knockout mice are determined in order to find whether the association of soluble NUM metalloproteinase with αvβ3-integrin is required for angiogenesis during tumor development. B6RV2 lymphoma cells are grown in a wild-type host containing normal appearing blood vessels that stained positively with an anti-CD31 (PECAM) antiserum (FIG. 4A, panel 1; 9 animals tested). This staining co-localizes with staining for αv-integrin (FIG. 4A, panel c). When the B6RV2 cells are grown in the Id1±Id3−/− host, most of the anti-CD3 I positive cells enclose no lumen at all but occasionally blood vessels are observed with very narrow lumens (FIG. 4A panel b; 9 animals tested). Importantly, the CD31 positive cells enclosing the lumen are completely negative for αv-integrin (FIG. 4A, panel d). Since αvβ3-integrin has been shown to recruit soluble MMP2 metalloproteinase, the expression of MMP2 on the surface of the tumor vasculature should also be affected. MMP2 staining is readily detected on the endothelial cells within tumors grown in a wild-type host (FIG. 4A, panel e) but is absent on the endothelial cells in the IdI±Id3-animals (FIG. 4A, panel f).

The deficiency of endothelial cells of the tumors grown in the Id knockout mice in metalloproteinase activity, is shown to be related to a thickening of the extracellular matrix around the forming blood vessels. Electron microscopic analysis of the endothelial cells present in the B6RV2 tumors grown in wild-type and Id1±Id3−/− mice show that a typical capillary within the B6RV2 tumor mass grown in wild-type animals endothelial cells (E) adjacent to a relatively narrow layer of ECM (FIG. 4b, panel a, between the arrows). In the Id1±Id3−/− animals however, the lumens of the capillaries are obstructed by what appeared to be cytoplasmic projections consistent with the gross histological data presented above (FIG. 4A, panel b). In addition, the ECM adjacent to the endothelial cells shows a marked thickening relative to that observed in the control animals. Taken together, these observations are consistent with the possibility that one defect in the Id1±Id3−/− endothelial cells is a failure to display active MMP2 on the surface of the endothelial cells resulting m an inability of the newly forming blood vessels to form a functional vascular network. This ultimately results in the regression of the B6RV2 tumor mass.

### Example 10

### Wild-Type Bone Marrow Cells and Restoration of Angiogenesis

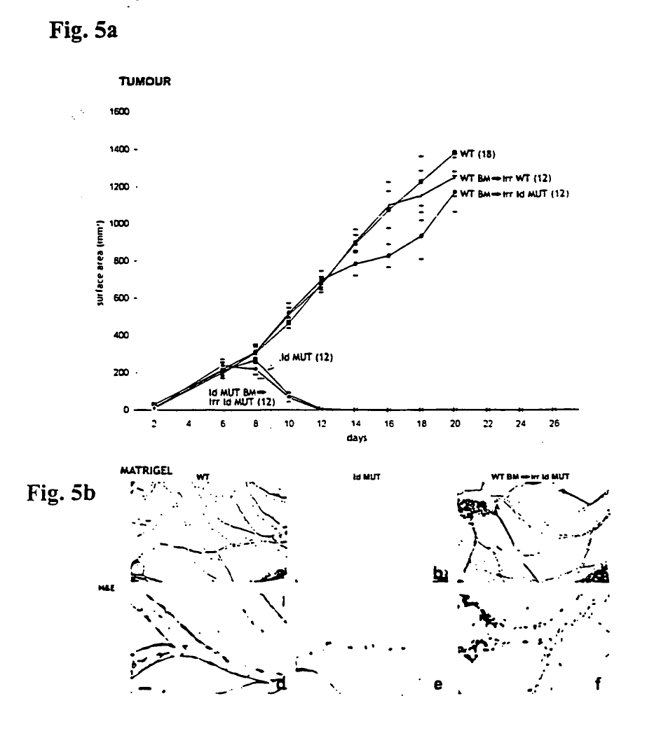

Mutant Id±Id3−/− mice were lethally irradiated, reconstituted with-donor wildtype bone marrow (BM) cells and the inoculated intradermally with B6RV2 lymphoma cells (FIG. 5a). In these reconstituted mice, tumor growth paralleled that observed in wild-type animals. Moreover, as in the wild-type animals, Id mutant mice engrafted with wild-type BM developed widespread mesenteric lymph node metastases and died prior to day 26. In contrast, rapid tumor regression was observed in non-transplanted Id mutant mice or irradiated Id mutant mice receiving Id mutant BM cells.

To determine whether the contribution of wild type donor BM in Id mutant mid 5 was restricted to tumor vasculature, vascular channel formation in VEGF loaded Matrigel plugs implanted subcutaneously in the abdominal cavity of the host animal. (FIG. 5B) Ten days after plug implantation, histological analysis showed no vascular channel formation in Id mutants (n=10) (FIG. 5B, b,e) while widespread vessel sprouting can be seen in the VEGF-loaded Matrigel plugs of the wild-type mice (FIG. 5B a,d). Notably, reconstitution of the Id mutant mice (n=12) with wild-type BM cells completely restored vascular channel formation. These date demonstrate that the wild-type BM cells are sufficient for the restoration of tumor growth and vascular channel formation in Id mutant host animals.

### Example 11

### Bone Marrow Derived Cell Recruitment and Angiogenesis

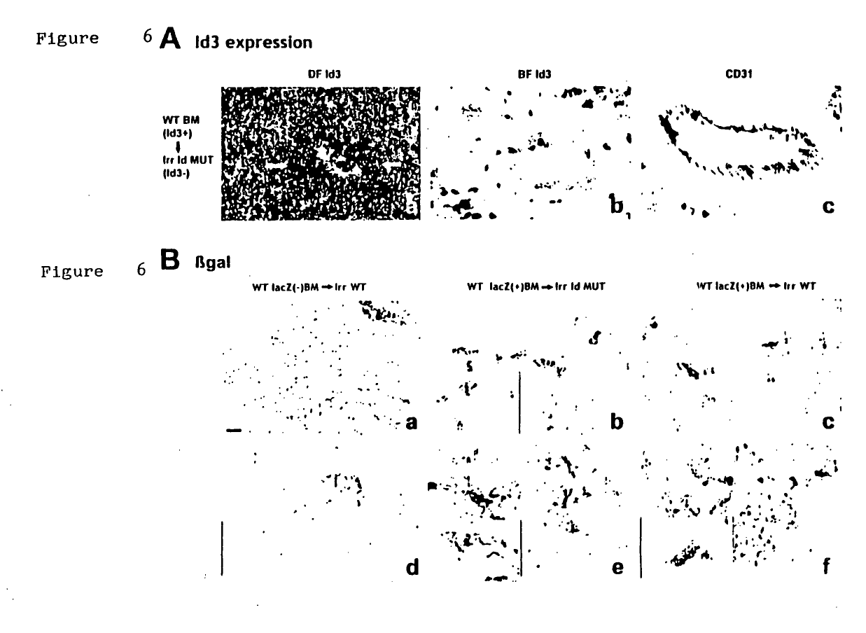

To assess whether the BM-derived cells were recruited to the neo-angiogenic site in B6RV2 tumors, RNA in situ hybridization for Id3 was performed (FIG. 6). Id3 expression was detected in tumor associated endothelial cells in irradiated Id1±Id3−/− mice (n=12) transplanted with donor Id1+/+Id3+/+ wild type BM (FIG. 6A a,b) Platelet endothelial cell adhesion marker (PECAM/CD31) co-expression with 1d3+ cells established the presence of mature blood vessels (FIG. 6A,c) To further confirm the contribution of BM-derived endothelial cells to the tumor vasculature, B6RV2 tumors were implanted into lethally irradiated Id mutant mice previously repopulated with BM derived from P-galactosidase (β-gal+) knock in mice (Rosa 26). Since Rosa 26 mice express 5-gal transgene in all tissues, LacZ staining of the tumor tissue can reveal donor derived BM cells readily. In tumors implanted for 14 days, a homogenous distribution of LacZ+ vessels was detected throughout the tumor tissue (Eosin stained) (FIG. 6B b,e) Greater than 959'o of the vessels expressing von Willebrand Factor (cWF) were characterized as Lac+ (FIG. 2b, inset). These results demonstrate that wild-type BM derived cells are incorporated extensively into vessels associated with B6RV2 tumors grown in Id mutant mice. The contribution of donor bone marrow cells to the tumor vasculature may have reflected the inability of the neighboring Id1 and/or Id3 deficient endothelial cells to be recruited to the neo-angiogenic vascular bed, thereby forcing the recruitment of donor BM cells. To examiner the relative contribution of preexisting and BM-derived CEFs in a more physiologically relevant model, BM from β-gal+ Rosa 26 mice was transplanted into lethally irradiated wild-type mice (n=12) and then challenged with tumor. As in tumor vessels of BM reconstituted Id mutant mice, LacZ stained blood vessels were detected throughout the vasculature of the B6RV2 tumor grafts in the wild type host animal (FIG. 6B c,f) In addition, LacZ staining was detected in all BM cells verifying a complete engraftment of the host BM, whereas the bone itself showed only eosinophilic staining (FIG. 6B e,f Insets) No LacZ staining was seen in either the BM or tumor tissue when BM cells of the wild type. β-gal(−) mice were transplanted (n=8) (FIG. 6B a,d Insets). Collectively, these results underscore the capacity of BM-derived cells to be mobilized to the tumor vascular bed and to contribute to the neo-angiogenic process.

### Example 12

### Association of Angiogenesis and VEGF-Induced MobaiuUion of Circulating Endothelial Precursor (CEP)

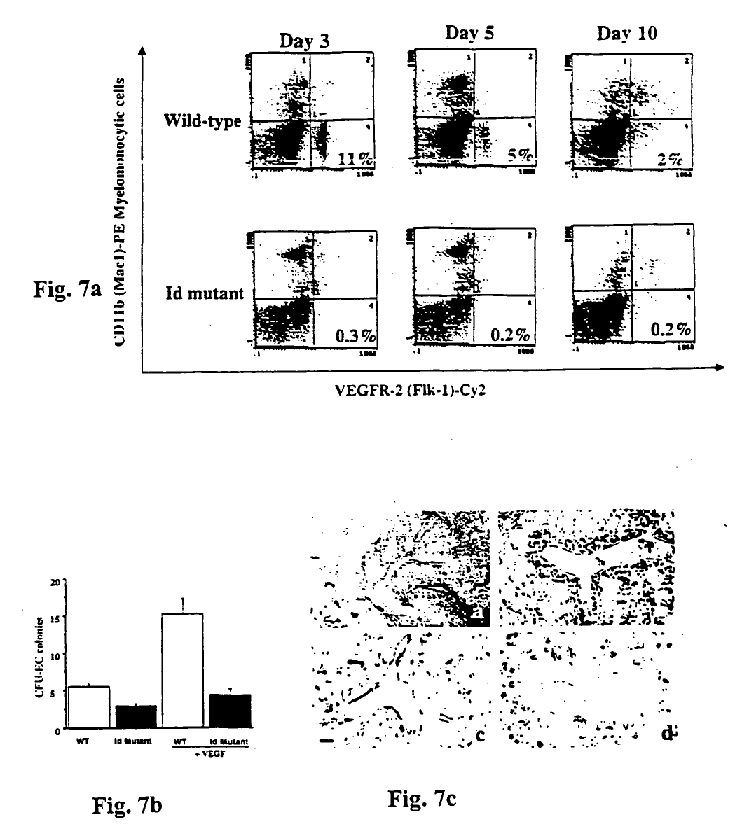

One explanation for the impairment of post natal angiogenesis in Id1+1−Id3-1mutant mice is the inability of CEPs to mobilize in response to VEGF1fi5 . To test this hypothesis, Id1±Id3−/− mutant and wild-type mice were injected with adenoviral vectors carrying VEGFISS transgene (AdVEGF₁₆₅) which allowed for the release of VEGF (average plasma level of 750 pglml) into the peripheral blood circulation at levels comparable to that observed in the presence of tumors. Elevation of plasma VEGF₁₆₅ levels in the wild type mice (n=6) induced mobilization to the peripheral circulation of a large percentage of mononuclear cells with the CEP potential expressing vascular endothelial growth factor receptor −2) (VEGFR2, Flk-1) that lacked the myelomonocytic marker (DC11b) (11% on day 3, 5% on day 5 and 0.2% on day 10) (FIG. 7A) in Id mutant nice (n=6). The VEGR2+ cells were most likely BM derived CEPs rather than mature endothelial cells since they were able to form VEGFR2 late outgrowth colonies of endothelial cells (CFU-EC) I in vitro cultures (FIG. 7B). These data support the notion that defective angiogenesis observed in Id1±Id3−/− mutant mice is a consequence of impaired VEGF-induced mobilization of CEPs.

The sustained release of VEGF in mice also induces mobilization of hematopoietic stem cells. To determine if transplantation of VEGF mobilized cells can rescue hematopoiesis and reconstitute angiogenesis in lethally irradiated Id mutant mice, βGal+Rosa 26 mice were injected with AdVEGF and mobilized cells harvested from the peripheral circulation were transplanted into lethally irradiated Id mutant mice. Similar to reconstitution with BM-derived cells. transplantation of VEGF-mobilized cells resulted in restart of angiogenesis and tumor growth of the implanted B6RV2 in Id mutant mice. LacZ stained cells can be seen in the blood vessels of the B6RV2 tumors (FIG. 7C a,b), demonstrating that VEGF-mobilized angio-competent CEPs are required for the induction of angiogenesis. Immunohistochemical analysis of day 2 tumors demonstrated the presence of vWF+IacZ+ vessels (FIG. 7C,c) decorated by VEGFR1+Lac+ mononuclear cells (FIG. 7Cd). In addition, virtually all the LacZ+ vessels also expressed VEGFRI (FIG. 7Cd) These data suggest that in the early phases of neo-angiogenesis recruitment of VEGF responsive BM-derived cells, composed of CEPs and VEGFR1+ precursor cells with morphologic features reminiscent of hematopoietic cells may be required to initiate the neo-angiogeneic process.

### Example 13

### Genotyping by PCR

Genomic DNA is obtained from mouse tail tips and yolk sacs as described by Hogan et al., J. Embryol. Exp. Morphol. 97:95-110 (1998) incorporated herein by its entirety. PCR analysis is performed with primers specific for the wild-type and targeted alleles. Prim sequences for Id1 are pr-22 (common oligonucleotide; 5′-CCTCAGCGACACAA GATGCGATCG-3′), pr-k4 (wild-type oligonudeotide; 5′-GGTTGCITITGAACGTTGTGAACC-3′) and pr-pgk (mutant oligonucleotide; 5′-GCACGAGACTAGTGAGACGTG3′). Primer sequences for Id3 are yz 151 (common oligonucleotide; 5′-GTMTFGAACATAGGTCTGCC-37, yz 170 (wild-type oligonucleotide; 5′-CACCGGGCTCAGCGCCTTCAT-3′),and yz 29 (mutant oligonucleotide; S′-TCGCAGCGCATCGCGF[CTA-3′). PCR cycling conditions are 90'C for 30s, 57° C. for 30s and 65°' C. for 3 min, for 40 cycles. The amplified PCR products are analyzed on 196 agarose gels to separate the wild-type (1.0 kb for Id1 and 2.0 kb for Id3) and targeted allele (o.8 kb for Id1 and 2.5 kb for Id3) fragments. PCR genotyping of acv-integrin-null nice is performed as described by Bader et al., Cell 95:507-519 (1998) incorporated herein by its entirety.

### Example 14

### Morphological and Histological Analysis

Embryos are obtained from timed pregnancies, with noon of the plug date defined as E0.5. The plug date is the date that embryos are removed. Embryos are fixed in 4% paraformaldehyde. Paraffin embedding is performed by dehydrating embryos through ethanol and Histoclear (National Diagnostics) before immersion in paraplast (Fisher Scientific). Sections of 6 or 7 micrometer are stained with hematoxylin and eosin (H&E).

Tumors, lung and enucleated eyes are fixed, processed and stained with H&E as described above. The entire lung per animal is sectioned and analyzed for metastases by two independent scorers. Blood vessels are counted in eight random 200× fields and results from two independent scorers are averaged.

### Example 15

### In situ Hybridization

Embryos and tumors are fixed in 4% paraformaldehyde and embedded in paraffin. 20 Sections (6 or 7 micrometer) are processed for in situ hybridization with (alpha-33P)UTP-labeled antisense RNA probes, as described by Manova et al, Dev. Dyn. 213: 293-308 (1998) incorporated herein in its entirety. Probe templates are provided by R Kagqama (MATH-1, MATH-2 and MATH-3) Akazawa et al, J. Biol. Chem 270:8730-8738 (1995). S. Tapscott (NeuroD2, NeuroD3) McCormick et al., Mol. Cell. Bio. 16:5792-5800 (1996), J. Lee (NeuroD) Lee et al., Science 268:836-844 (1995),E. Lai (BF-1)20,E. Boncinelli (Emx2) Simonson et al., Nucleic Acids Res. 21: 5767-5774 (1993), P Gauss (Pax6) Walther et al., Development 113:1435-1449 (1991), J. Rubenstein (DIx2) Price et aL, Nature 351:748-751(1991), and A. McMahon (Shh) (Shimamura et al., Development 121:3923-3933 (1995)).

### Example 16

### Whole-Mount Immunohistochemistry

Embryos are processed as described by Winnier et al., Genes Dev. 11, 926-940 (1997). Embryos are incubated with primary antibody WC13.3 rat monoclonal antimouse PECAM-1 antibody; Pharmingen). Samples are incubated with biotinylated anti-rat antibody (Vector), and then with peroxidase-conjugated avidin (Vector). For colour detection, NiCI2 and DAB are added to embryos

### Example 17

### Immunohistochemistry

For Ki67 immunohistochemistry, tissue antigens are unmasked and sections are incubated with monoclonal anti-muse K167 antibody (NCL-Ki67-MMI; Novocastra Laboratories), followed by biotinylated anti-mouse antibody. The Histomouse-Sp Kit (Zymed Laboratories) is used. For all other antigens the following antibodies are used: MAP2 and p16, anti-rat MAP2 (clone MM-2; Sigma) or monoclonal anti-p16 (Santa Cruz) antibody; PECAM (CD31), MEC 13.3 monoclonal anti-mouse PECAM-1 antibody (Pharmingen); laminin, polyclonal anti-laminin antibody (Sigma); VEGF, and-VEGF (Santa Cniz), Flk 1, anu-Flk I (Sigma); smooth muscle alpha-action-anti-alpha-action (Sigma); αv-integrin antibody (Chemicon); MI—T2 (gelatinase-A), polyclonal anti-mouse NIMP-2 antibody (Sigma). All sections are then incubated with the appropriate biotinylated secondary antibodies (Vector).

### Example 18

### Cell Lines

B6RV2, a murine leukemia/lymphoma cell line generated at Memorial Sloan-Kettering Cancer Center and LLC, obtained from American Type Culture Collection, are used. B-CA, breast carcinoma cell line, is established from tumors generated by crossing Id1-nuD mice and mammary tumor virus-polyoma virus knockout mice. All lines are maintained in DMEM with 10% fetal calf serum.

### Example 19

### Tumor Implantation In a Murine Model

Roughly 2×10⁷ cells of each tumor cell line are injected intradermally in the right lower abdomen Surface area is measured by two independent scorers (Dial Caliper, and Scianceware). For intravenous injection of tumor, 2×10⁶ LCC cells are injected in the tall veins of anesthetized mice (2.59b Avertin). For the eye implantation studies, animals are anesthetized with 2.596 Averiin and proparicane hydrochloride ophthalmic solution (o.5′o). Roughly 2×10⁶ B6RV2, LLC cells or media alone are injected with a Hamilton syringe and needle with the assistance of a dissecting microscope (Zeiss Olympus) into the corneal layers of the eye.

### Example 20

### Electron Microscopy

Tissues are processed with “yellowfix” (2.536 glutaraldehyde, 4% parafomaldehyde, 0.02% picric acid in 0.1 M Na-cacodylate). The samples are post-fixed with 1% osmium tetroxide-1.5% ferricyanide, dehydrated in ethanol and infiltrated with Spurr's Resin. Tissue blocks are trimmed with a diatome diamond knife (Diatome USA) on RMA NMOO ultramicrotome. Sections are contrasted with lead citrate and viewed on a JEOL IOOCX-I[l electron microscope.

### Example 21

### Preparation of Knockout Constructs

The DNA sequence to be used in producing the knockout construct is digested with a particular restriction enzyme selected to cut at a specific location(s) such that a new DNA sequence encoding, for example, a marker gene to be inserted in the proper position within this DNA sequence. The proper position for marker gene insertion depends on factors such as the restriction sites in the sequence to be cut, and whether an exon sequence or a promoter sequence, or both are to be interrupted (i.e., the precise location of insertion necessary to inhibit promoter function or to inhibit synthesis of the native exon). In some cases, it is desirable to actually remove a portion or even all of one or more exons of the Id1 or Id3 gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size is removed.

The marker gene is any nucleic acid sequence that is detectable and/or assayable. However, typically it is an antibiotic resistance gene or other gene whose expression or presence in the genome is easily detected. The marker gene is usually operably linked to its own promoter or to another strong promoter from any source that will be active or can easily be activated in the cell into which it is inserted. However, the marker gene need not have its own promoter attached, as it may be transcribed using the promoter of the gene to be suppressed. In addition, the marker gene normally has a polyA sequence attached to the 3′end of the gene. This sequence serves to terminate transcription of the gene. Preferred marker genes are any antibiotic resistance gene such as neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan and described in Sambrook et al., supra. The ends of the DNA fragments to be ligated must be compatible. This is achieved by either cutting ail fragments with enzymes that generate compatible ends, or by blunting the ends prior to ligation. Blunt ending of the sequence is achieved using, methods well known in the art, such as for example by the use of Klenow fragment (DNA polymerase I) to fill in sticky ends. The ligated knockout construct is inserted directly into embryonic stem cells, or it is first placed into a suitable vector for amplification prior to insertion. Preferred vectors are those that are rapidly amplified in bacterial cells such as, viral vectors or pBluescript 11 SK vector Stratagene, San Diego, Calif.) or pGEM7 (Promega Corp., Madison, Wis.).

### Example 22

### Bone Marrow Transplantation for Tumour and Matrigel Plug Assays

Mice were genotyped by polymerase chain reaction (PCR) of tail DNA as described (Lyden, D. et al. Nature 401, 670-7 (1999)). Id mutant (Id1±Id3−/−) and wild-type C57B1/6/SvI29 mice were lethally irradiated with 950 rads. Approximately, 1×10⁶ β-gal negative or positive (Rosa 26 mice) BM cells were injected into tail veins of irradiated recipient mice. Following four weeks, allowing for BM reconstitution, mice were injected intradermally with either 2×10⁷ B6RV2 murine lymphoma cells (established at Memorial Sloan-Kettering Cancer Center) or one ml of iced Matrigel (BectonDickinson) and admixed with VEGF (Peprotech, 10 μg/ml.) and heparin (Sigma, 100 fcglmL) into the right lower abdomen. For the tumour, surface area was scored by three independent observers (Dial Caliper, Science Ware).

### Example 23

### Histological Analysis, Immunohistochemistry, In situ Hybridization in Bone Marrow Recipient Mice

Tumour tissue and Matrigei plugs were fixed is 4% paraformaldehyde for four hours. Paraffin embedding was performed by dehydrating tissue and plugs through ethanol and Histoclear (National Diagnostics) before immersion in paraplast (Fisher Scientific). Sections of 8 pm were stained with hematoxylin and eosin and antibodies to PECAM (CD31) (MEC 13.3 monoclonal antibody (mAb) anti-mouse PECAM-1 antibody (Pharmagen), vWF (combined primary and biotinylated secondary antibody (Dako), and VEGFRI (Flt-1, biotinylated mAB, clone ]NO-1, ImClone Systems) were used. For in situ hybridization, sections were hybridized to (a-³³P) UTP labeled anti-sense RNA probes as described (Lyden,D. et al. Nature 401, 670-7 (1999)).

### Example 24

### β galactosldase (LacZ) Staining in Bone Marrow Recipient Mice

Tumour tissue and femoral bones, split in two to expose marrow, were fixed in 4% paraformaldehyde for two hours. The tumour tissues were further dissected into small pieces and the marrow was flushed in whole from the bone. The samples were washed in PBS and PBS containing washing buffer solution (2 mM MgCl₂, 5 mM EDTA, 0.01% sodium deoxycholate, 0.02% NP-40) and stained in fresh x-gal solution at 37° C. overnight according to methods previously described¹⁶. The X-gal stained tumour and BM then were embedded in paraffin, sectioned, and counter-stained with eosin to visualize LacZ negative tissue.

### Example 25

### VEGF-Induced Mobfizadon

Id mutant or wild-type mice were injected intravenously with 10⁸ MOI of E1-E4+ AdVEGF and as control same dosage of AdNull as previously described^{13,17}. Mobilized peripheral blood mononuclear cells (PBMC) from AdVEGF,ss or AdNull treated mice were collected by orbital bleeding and stained with FITC-conjugated anti-VEGFR2 (clone DC101) mAb and MI lb (Macl, myeloid lineages)-Phycoerythrin. Stained cells (1×10⁴) were analyzed on a Coulter Elite flow cytometer to determine the representative percentages of positive populations in PBMCs. VEGF plasma levels were measured at the time of orbital bleeding. ¹³For quantification of CEPS with early and late outgrowth potential, 5×10⁴ mobilized PBMCs obtained from AdVEGF₁₆₅ or AdNull treated Id mutant or wild-type mice on a day o to day 21 and plated in the presence of modified endothelial growth medium on collagen/fibronectin coated plastic dishes, as previously described^{11,12,13}. Endothelial growth medium consisted of X-vivo 20 serum free medium (BioWhittaker), supplemented with VEGF (10 ng/ml), basic FGF (5 ng/ml), heparin 10 units/ml, and endothelial growth supplement (Collaborative Research). Endothelial colonies (CFU EC) were identified and quantified by co-staining with DiI-Ac-LDL metabolic labeling and vWF immunostaining^{11,12}. Colonies that formed within the first three days (early outgrowth) and colonies that formed by 14 days (late outgrowth) were quantified by Dil-Ac-LDl labeling after the start of culture (mcan±SEM. Transplantation of VEGF mobilized PBMC from wild-type Rosa mice into lethally irradiated Id mutant were performed as described above. A total of five million VEGF mobilized PBMC from day 3 and 5 were collected, Ficolled and transplanted by tail-vein injections into lethally irradiated hosts.

### LIST OF REFERENCES

1. Norton et al, Trends Cell Biol. 8(2):58-65 (1998)
2. Deed et al., J. Bio. Chem 27:8278-8286 (1998)
3. Ogata et al., Proc. Natl. Acad Sci. USA go: 9219-9222 (1993)
4. Neuman et al., Dev. Biol. 160:186-195 (1993)
5. Heemskerk, et al., J. Exp. Med 186:1597-1602 (1997)
6. Martinsen et al., Science 281:988-991(1998)
7. Katagiri et al., J. Cell. Biol. 127:1755-1766 (1994)
8. Israel et al., Cancer Res. 1(59):1726-1730 (1999)
9. Maruyanza et al., Am. J. Pathol. 155(3):815-22 (1999)
10. Yan et al., Mol. Cell Biol. 17(12): 7317-27 (1997)
11. Duncan et al., Dev. Biol. 154:1-10 (1992)
12. Ellneier et al., Dev. Dyn. 203:163-173 (1995)
13. Jen et al., Dev. Dyn. 208:92-106 (1997)
14. Lyden et al., Nature 401:670-677 (1999)
15. Risau et al., Nature 386: 671-674 (1997)
16. Pfeffer et al., Cell 73:457-467 (1993)
17. Fung Leung et al., Cell 65:443-449 (1991); J. Exp. Med, 174:1425-1429 (1991)
18. Xuan et al., Neuron 14:1141-1152 (1995)
19. Price et al., Nature 351: 748-751(1991)
20. Simeone et al., EMBO J.12:2735-2747 (1993)
21. Walther et al., Development 113:1435-14!19 (1991)
22. Shimamura et al., Development 121:3923-3933 (1995)
23. Hogan et al., J. Einbnol. Exp. Morphol. 97:95-110 (1998)
24. Bader et al, Cell 95:507-519 (1998)
25. Manova et al., Dev. Dyn. 213: 293-308 (1998)
26. Winnier et al, Genes Dev. 11, 926-940 (1997)
27. Akazawa et al., J. Biol. Chen 270:8730-8738 (1995)
28. McCmmick et al., Mol. Cell. Bio. 16:5792-5800 (1996)
29. Lee et al., Science 268: 836-844 (1995)
30. Simonson et al., Nucleic Acids Res. 21: 5767-5774 (1993)
31. Walther et al, Development 113:1435-1449 (1991)
32. Price et al., Nature 351:748-751(1991)
33. Sambrook et al., Molecular Cloning: A Laboratory Manual, Cold Spring Harbor Laboratory Press, Cold Spring Harbor, N.Y. (1989)
34. Teratocarcinoinas and Embryonic Stem Cells: A Practical Approach, E J. Robertson, ed. IRL Press, Washington, D.C. (1987)
35. Bradley et al., Current Topics in Devel. Biol., 20:357-371(1986)
36. Hogan et al., Manipulating the Mouse Embryo: A Laboratory Manual, Cold Spring Harbor Laboratory Press, Cold Spring Harbor, N.Y. (1986)
37. Maniotis et al., Am. J. Pathol. 155(3): 739-52 (1999)
38. Ogata et al., Proc. Natl. Acad Sci. USA 90: 9219-9222 (1993),
39. Holash, D. & Folkman, J. Cell 86, 353-64 (1996)
40. Yankopoulos, GI). et al Nature 407242-8 (2000)
41. Folkman, J. et al., Nature 339,58-61(1989)
42. Raffi, S, J. Clip. Invest 10517-19 (2000)
43. Asahara. T. et al, Science, 275, 964-7 (199'1]
44. Kalka, C, et al., Oric, Nat'l Acad Sci USA 973422-7 (2000)
45. Takahashi, T. et al., Nat Med B5, 434-8 (1999)
46. Peichev, M. et al. Blood 95 952-8 (Feb. 1, 2000)

What is claimed is:

1. A transgenic knockout mouse comprising a disruption of at least one allele of both of its Id1 and Id3 genes, where at least one of the Id1 and Id3 genes is heterozygous, and the mouse exhibits a prevention or reduction of tumor growth.

2. The transgenic knockout mouse of claim 1, where the disruption in the genes are one of the following: Id1±Id3±, Id1±Id3−/− or Id1−/−Id3±.

3. The knockout mammal of claim 1 wherein said construct contains an oncogene or a proto-oncogene.

4. The knockout mammal of claim 1 wherein said proliferative cell disorder is induced in said mammal via a tumor xenograft.

5. The knockout mammal of claim 2, wherein said mammal is heterozygous for Id1 gene and homozygous for Id3 gene.

6. The knockout mammal of claim 1, wherein said mammal is homozygous for Id1 gene and heterozygous for Id3 gene.

7. The knockout mammal of claim 5, wherein said mammal is Id1± and Id3−/−.

8. The knockout mammal of claim 1, wherein said cell proliferative disorder is cancer.

9. The knockout mammal of claim 8, wherein said cancer comprises, breast cancer, lung cancer, lymphoma, or a combination thereof.

10. The knockout mammal of claim 9, wherein said cancer do not metastasize or vascularize in the body.

11. The knockout mammal of claim 8, wherein expression of integrin and metalloproteinase are substantially reduced in said cell proliferative disorder.

12. The knockout mammal of claim 1, wherein said mammal is a mouse.

13. The knockout mammal of claim 1, wherein said disruption affects transcription and/or translation of a polynucleotide encoding at least one gene product of Id1, Id3, or both.

14. The knockout mammal of claim 1 wherein said disruption affects post-transitional activity of said at least one gene product of Id1, Id3, or both.

15. A method of preventing, ameliorating, or treating a cell proliferative disorder in a subject having an inappropriate or aberrant Id product, comprising administering to said subject a physiologically effective amount of an agent capable of interaction with at least one inhibitor of differentiation (Id) gene product in the body of said subject.

16. The method of claim 15 wherein said cell proliferative disorder comprises angiogenesis.

17. The method of claim 16 wherein said angiogenesis is a tumor angiogenesis.

18. The method of claim 15 wherein said interaction produces an antagonistic, or agonistic effect on expression, activity, or both of said at least one Id gene product.

19. The method of claim 15 wherein said agent comprises proteins, peptides, sense or antisense nucleic acid molecules, small molecules, or a combination thereof.

20. The method of claim 17 wherein said administration additionally comprises one or more anti-cancer agents.

21. A method to screen agents for use in treating neurological and/or cell proliferative disorders, comprising the steps of:
a) incubating mammalian cells in the presence and absence of a test agent,
b) determining levels of expression and or activity of at least one gene product of Id1, Id3, or both in said cells incubated in the presence and absence of said test agent; and; and,
c) selecting an agent that interacts with expression and/or activity of said at least one gene product of Id1, Id3, or both, as compared to control, for use in treating said neurological and/or cell proliferative disorders.

22. The method of claim 21 wherein said interaction is agonistic, antagonistic, or both.

23. A method to screen agents useful in treating, neurological and/or cell proliferative disorders, comprising the steps of:
a) administering a test agent to a mammal;
b) determining level of expression and/or activity of at least one gene product of Id1, Id3, or both; in the presence and absence of a test agent;
c) selecting an agent that affects expression and/or activity of said at least one gene product of Id1, Id3, or both, as compared to control, for use in treating said neurological and/or cell proliferative disorders.

24. The method of claim 23 wherein said mammal has been altered to contain a disruption in at least one and at most three alleles of inhibitor of differentiation gene, Id1 and Id3 genes.

25. The method of claim 23 wherein said mammal is being genetically transformed with a construct that is capable of producing cancer spontaneously in said mammal.

26. The method of claim 23 wherein said mammal is implanted with one or more tumor xenograft.

27. The method of claim 26 wherein said tumor xenograft comprises xenograft of lymphoma, breast cancer, lung cancer, or a combination thereof.

28. A diagnostic method for determining whether a subject has, or is at risk for developing, a neurological and/or an angiogenic disorder comprising the steps of:
a) obtaining a sample from said subject;
b) determining level of expression and/or activity of at least one gene product of Id1, Id3 or both; in said subject; and
c) detecting presence or absence of a genetic mutation in said subject, wherein said genetic mutation results in inappropriate or aberrant one or more Id product activity and/or expression, wherein said genetic mutation identifies a subject that has, or is at risk for developing, a neurogenic or cell proliferative disorder or disease.

29. A method for reducing or inhibiting, tumor vasculature in a mammal comprising; introducing a cell population to said individual, said cell population is transformed with a polynucleotide molecule encoding and expressing in the body of said individual a biologically effective amount of an antagonizer of one or more gene products of Id1, Id3, or both.

30. The method of claim 29 wherein said antagonizer is tetracycline.

31. A diagnostic test it for detecting the presence or absence of a genetic mutation in a subject resulting in an inappropriate or aberrant one or more Id product activity and/or expression, comprising:
(a) a probe which specifically hybridizes to one or more Id gene, or gene products;
(b) a reagent means for detecting said hybridization; wherein the probe and reagent means are each present in amounts effective to perform the hybridization assay.

32. The diagnostic test kit of claim 31, wherein said probe is an antibody.
